(19) United States
(12) Patent Application Publication
(10) Pub. No.: US 2002/0139710 A1
(43) Pub. Date: Oct. 3, 2002

(54) Control carbonyl concentration in diolefinic products using hydroxyamine sulfate as an extraction aid

(76) Inventors: John F. Martin, Conroe, TX (US); Sherif Eldin, Houston, TX (US); Terry Kelly, Prairieville, LA (US)

Correspondence Address: Hercules Incorporated, 1313 North Market Street, Wilmington, DE, 19894-0001, US

(21) Appl. No.: 09/775,751

(22) Filed: Feb. 2, 2001

(51) Int. Cl. C07C 7/10; C07C 7/152; C07C 7/17; C10G 9/12

(52) U.S. Cl. 208/48 R; 208/48 AA; 208/289; 585/860; 585/950; 585/833

(57) ABSTRACT

The present invention is directed to the use of hydroxylamine, its acid salts or mixtures thereof to increase carbonyl extraction during the basic washing of hydrocarbons containing oxygenated compounds. More specifically, oxygenated compounds such as carbonyl containing organics are typically an impurity and have a tendency to polymerize, producing fouling elements during processing. The hydroxylamine and its salts are quite effective for increasing the extraction of the carbonyl impurities during the caustic washing of hydrocarbon streams.

### FIELD OF THE INVENTION

This invention relates to methods for controlling the carbonyl compound concentration in diolefinic hydrocarbon streams. More specifically, this invention is directed to the use of hydroxylamine, its acid salts or mixtures thereof to aid in carbonyl extraction during the basic washing of hydrocarbons containing oxygenated compounds.

### BACKGROUND OF THE INVENTION

This invention relates to improving the carbonyl removal of a basic solution which is in contact with a gaseous or liquid hydrocarbon stream.

In cracking operations (pyrolysis) such as in the cracking (pyrolysis) of ethane, propane, gas oils and naphthas to olefins, oxygenated compounds, including carbonyl compounds, are formed. The amount of carbonyl compounds, such as aldehydes and ketones, formed in such an operation can vary widely, but is typically about 1-100 ppm in the gas stream with concentrations as high as 1000 ppm occasionally being encountered because of the utilization of the various feedstocks and cracking temperatures. A portion of these impurities are generally removed when the gas stream is passed through a basic wash (pH>7) to remove acidic components such as hydrogen sulfide and carbon dioxide. The typical basic wash systems include amine acid gas scrubber (e.g., MEA, DEA, isopropyl amine, butyl amine, etc.) and caustic wash systems. Unfortunately, some amine scrubbing units require a shutdown period for maintenance and the remaining units (caustic towers) must bear the burden of acid gas and carbonyl removal. Unacceptable removal of carbonyl compounds from the hydrocarbon streams can lead to off-spec product, fouling of the caustic tower and fouling of downstream equipment such as a spent caustic steam stripper.

In U.S. Pat. No. 4,673,489, hydroxylamine, its acid salts or mixtures thereof are used to prevent fouling during basic washing or hydrocarbons containing oxygenated compounds. At column 3, lines 18-20, the patent teaches that a molar ratio of hydroxylamine to oxygenated or carbonyl compound of 1:1 or greater is required.

U.S. Pat No. 4,952,301 discloses a method for inhibiting the formation of polymeric based fouling formed during the caustic washing of hydrocarbons comprising adding an effective amount of an ethylenediamine compound to the caustic wash system.

U.S. Pat. Nos. 3,336,414 and 3,308,201 disclose processes utilizing aqueous caustic washes (pH>10) of carbonyl-contaminated hydrocarbons to remove carbonyl compounds.

In U.S. Pat. No. 3,281,489, carbonyl compounds, aldehydes, are removed from a butadiene stream (obtained from the pyrolysis of saturated hydrocarbons) by selective hydrogenation to reduce some of the carbonyls. This procedure is followed by caustic washing to remove substantially all of the remaining carbonyl compounds. If caustic washing is performed before the hydrogenation, carbonyl polymeric materials from aldol condensation foul the process equipment. U.S. Pat. No. 3,801,669 discloses the use of Portland cement to extract carbonyl compounds from hydrocarbon streams.

At acid pH, extraction of carbonyl compounds from hydrocarbon streams by aqueous solutions of hydrazine compounds is reported in U.S. Pat. No. 3,793,187. The carbonyl compounds need to be extracted because they have an inhibiting effect on further processing steps. Only extractions of liquid systems are contemplated in this patent.

In U.S. Pat. No. 3,535,399, carbonyl compounds are removed from gaseous hydrocarbon streams by contacting the streams with an aqueous solution of sodium hydroxide and urea. The caustic removes acid materials and the urea complexes with the carbonyl compounds to form aldehyde-urea or ketone-urea resins that are reportedly entrained in the aqueous solution.

Substituted hydroxylamine compounds such as N,N-diethylhydroxylamine are used to prevent polymerization of unsaturated aldehydes in dilute alcohol solutions, but not of the neat unsaturated aldehydes in U.S. Pat. No. 3,849,498.

German Pat. No. 1,072,607 discloses that polystyrene-based cation exchange resins can be treated with a solution of hydroxlyamine hydrochloride to remove carbonyl compounds from sulfite liquor.

French Pat. No. 1,546,472 discloses a procedure of treating a carbonyl-contaminated glycerol with an acid and 2,4-dinitro-phenyl hydrazine.

### SUMMARY OF THE INVENTION

Carbonyl extraction is aided in basic (pH 7 and higher) wash systems of the type adapted to remove impurities from liquid or gas phase hydrocarbon mediums by adding to the wash system a hydroxylamine, its acid salts or mixtures thereof.

After reviewing the problems associated with carbonyl contamination of hydrocarbons, particularly the gaseous olefins derived from pyrolytic cracking, it was apparent to the present inventors that the cracking industry required a treatment which would improve the extraction of carbonyls during the basic wash of hydrocarbons. Most desirably, the treatment would be such that it would operate effectively in the highly basic wash to alleviate the potential problems due to the carbonyl compounds without the formation of other solid materials that had to be removed. The treatment not only had to be effective but also cost-effective.

The present inventors discovered a method of improving the extraction of carbonyl compounds during the basic wash and in particular the caustic wash of hydrocarbons containing oxygenated compounds, and in particular the gaseous olefins containing carbonyl compounds. The latter carbonyl compounds under alkaline conditions undergo in many instances aldol condensation reactions to produce polymeric materials which deposit on the equipment and in particular plug pipes and packing or trays in the caustic wash tower and downstream spent caustic processing.

The inventive method is particularly appropriate for the basic washing process which follows the pyrolytic cracking of such hydrocarbons as ethane, propane, butane, naphtha, and gas oils and mixtures thereof to produce the corresponding gaseous ethylene, propylene, butadiene and the like, containing the carbonyl as well as other contaminants.

Generally the basic washing entails contacting in wash towers an aqueous basic solution with the gaseous olefins to remove any hydrogen sulfide, carbon dioxide and other oxygenated compounds. As earlier discussed, the incomplete or unacceptable removal of the oxygenated compounds, such as carbonyl compounds, can lead to contaminated product and fouling of downstream equipment.

The method entails assuring that the basic wash takes place in the presence of the hydroxylamine compounds thereof.

### DETAILS OF INVENTION

The hydroxylamine compounds of the invention can be used in the form of their hydrates, or salts wherein said salt is derived from a mineral acid such as sulfuric acid, hydrochloric acid, nitric acid, etc., or salts wherein said salt is derived from an organic acid such as acetic acid, propanoic acid, and the like. The acid portions of the salts will be neutralized by the base of the system and since only small portions of the acid salts are to be used, very little of the base will be used in the neutralization.

These extraction aides can be added to the caustic tower as neat materials or as solutions. The preferred method of addition is as an aqueous solution with 2 to 50 weight percent inhibitor present, so that accurate metering of the inhibitor to the tower can be achieved. The fouling inhibitors can be used in a continuous or batch process.

It is theorized that the extraction aid is present in the recirculating caustic as free hydroxylamine (NH₂OH) and is preferentially reacting with carbonyl compounds as they pass through the liquid recirculating aqueous caustic solution. The reaction product formed is non-volatile and is soluble in the recirculating caustic solutions. Additional amounts of carbonyl compounds are removed from the cracked gas with hydroxylamine than would otherwise not be removed by caustic scrubbing/contact alone. The extraction aid should be added in amount to effectively reach the desired extraction efficiency. In general, the extraction aid is added in a ratio of hydroxylamine to oxygenated or carbonyl compound of about 0.001:1 to about 0.99:1 with a preferred ratio of about 0.001:1 to about 0.6:1.

This product would be added to the wash (recirculating caustic solutions or fresh caustic makeup) in quantities to assure that desired extraction efficiency is achieved. Treatment ranges of from about 1 to 50,000 parts of product per million of wash solution could be utilized.

The invention will be further illustrated by the following examples which are intended merely for purposes of illustration and are not to be regarded as limiting the scope of the invention or the manner in which it may be practiced.

### EXAMPLES

In a North American ethylene plant, pyrolytic cracking gases are first contacted with lean monoethanolamine (MEA) solvent to remove the majority of acid gases (H2S and CO2) and oxygenated or carbonyl compounds. the cracked gas leaving the MEA absorber column then contact recirculating caustic in caustic wash towers. The caustic wash towers remove the remaining trace amounts of acid gases and carbonyl compounds without any treatment. However, the MEA system fouls with aldol condensation polymers of carbonyls and is shut down for cleaning periodically. When the MEA system is shutdown, the cracked gases are by-passed around the MEA unit and sent directly to the caustic wash towers for complete acid gas removal. The following trials in Table 1 were run on separate days. The trials were conducted while the upstream MEA system was shutdown for cleaning. Hydroxylamine sulfate was added to the aqueous caustic wash of two separately operating caustic towers. The influent carbonyl concentration of the gas and carbonyl concentration of the crude butadiene product stream was measured by gas chromatography and is compared.

TABLE 1

| | | hydroxyl- | | | |
|---|---|---|---|---|---|
| | CH₃CHO | amine | CH₃CHO | CH₃CHO | Molar Ratio |
| | (lbs./day) | sulfate | in Product | Removal | (HA: |
| Trial | in gas | (lbs./day) | (lbs./day) | (%) | CH₃CHO) |
| 1 | 542.88 | 864.0 | 7.752 | 98.572 | 0.434 |
| 2 | 508.59 | 936.0 | 20.808 | 95.909 | 0.516 |
| 3 | 516.83 | 936.0 | 7.776 | 98.495 | 0.494 |
| 4 | 602.78 | 864.0 | 8.352 | 98.614 | 0.391 |
| 5 | 491.11 | 957.6 | 7.205 | 98.533 | 0.532 |

Trials 1-5 illustrate the efficacy of the invention. When trials approximately identical to the above were run without the presence of hydroxylamine, it was reported that insufficient extraction was observed and the allowable carbonyl specification in the crude butadiene product was significantly exceeded.

While this invention has been described with respect to particular embodiments thereof, it is apparent that numerous other forms and modifications of this invention will be obvious to those skilled in the art. The appended claims and this invention generally should be construed to cover all such obvious forms and modifications which are within the true spirit and scope of the present invention.

What I claim is:

1. A method for increasing the extraction of oxygenated compounds during the basic washing of hydrocarbons contaminated with oxygenated compounds which comprises performing the wash of the hydrocarbon in the presence of sufficient amount for increasing extraction of oxygenated compounds of hydroxylamine, of the formula NH₂OH or an acid salt or mixtures thereof.

2. A method according to claim 1 wherein the hydrocarbon being washed is produced by the pyrolytic cracking of other hydrocarbons.

3. A method according to claim 2 wherein said other hydrocarbon is ethane, propane, butane, naphtha, gas oils or mixtures thereof.

4. A method according to claim 3 wherein the hydrocarbon being washed contains an olefin contaminated with oxygenated compound impurities.

5. A method according to claim 4 wherein the hydrocarbon being washed is in a gaseous state.

6. A method according to claim 5 wherein the oxygenated compounds are comprised primarily of carbonyl compounds.

7. A method according to claim 6 wherein the carbonyl compounds are aldehydes, ketones or mixtures thereof.

8. A method according to claims 6 or 7 wherein the hydroxylamine or acid salt thereof is added to the basic wash in an amount representing a molar ratio of said hydroxylamine or acid salt thereof to said carbonyl of less than 1:1.

9. A method according to claim 8 wherein the molar ratio is 0.001:1 to 0.99:1.

10. A method according to claim 8 wherein the ratio is 0.01:1 to 0.6:1.

11. A method according to claims 9 or 10 wherein the hydroxylamine or acid salt thereof is added to a recirculating caustic solution or fresh caustic makeup.

12. A method of increasing the extraction of oxygenated compounds during the basic wash of pyrolytically produced olefin contaminated with at least one carbonyl compound, which comprises adding to said wash during said basic wash of said olefin a sufficient amount for increasing the extraction of said at least one carbonyl compound of hydroxylamine, of the formula NH₂OH or an acidic salt or mixtures thereof.

13. A method according to claim 12 wherein said olefin with carbonyl ompounds is in a gaseous state.

14. A method according to claim 13 wherein said carbonyl compound is an aldehyde, ketone or mixture thereof.

15. A method according to claim 14 wherein said hydroxylamine or acid salt thereof is present in an amount which represents a molar ratio of such to the carbonyl compound is less than 1:1.

16. A method according to claim 15 wherein the molar ratio is from about 0.001:1 to 0.99:1.

17. A method according to claim 15 wherein the molar ratio is from about 0.01:1 to 0.6:1.

18. A method according to claim 13, 14 or 15 wherein the hydroxylamine is an acid salt form or is hydroxylamine sulfate.

19. A method according to claim 13, 14 or 15 wherein the hydroxylamine is in salt form and is hydroxylamine chloride.

20. A method according to claim 13, 14 or 15 wherein hydroxylamine is added to said caustic wash.

21. A method according to claim 20 wherein the caustic wash is a recirculating caustic solution stream or a fresh caustic makeup stream.
